(19) United States
(12) Patent Application Publication
(10) Pub. No.: US 2010/0049007 A1
(43) Pub. Date: Feb. 25, 2010

(54) Integrated physiological sensor apparatus and system

(76) Inventors: Bernhard B. Sterling, Danville, CA (US); Andrew R. Lawrence, Eagan, MN (US); Gregory I. Voss, Solana Beach, CA (US); James M. Perry, Nashville, TN (US); Rankin A. Clinton, III, Franklin, TN (US)

Correspondence Address: Ralph C. Francis;Francis Law Group, 1942 Embarcadero, Oakland, CA, 94606, US

(21) Appl. No.: 12/229,080

(22) Filed: Aug. 20, 2008

(51) Int. Cl. A61B 5/00

(52) U.S. Cl. 600/301

(57) ABSTRACT

A physiological sensor apparatus, system and method for determining a physiological characteristic, comprising providing at least one physiological sensor that is adapted to measure at least one physiological characteristic at a target measurement site on a subject's body, heating an extended tissue region on the subject's body, whereby blood perfusion of the tissue region is enhanced, and measuring at least one physiological characteristic at the target measurement site with the physiological sensor during or within a predetermined period after heating the extended tissue region. In one embodiment, the sensor system includes at least one temperature algorithm that is adapted to adjust the heat applied to the extended tissue region based on the body's response to the heat stimuli.

### FIELD OF THE PRESENT INVENTION

The present invention relates to the field of physiological sensors. More specifically, the invention relates to an integrated physiological sensor apparatus and system having heating means to enhance blood perfusion and algorithms to control the heating means and optimize blood perfusion.

### BACKGROUND OF THE INVENTION

It is well known in the art that pulse oximetry is based on the principle that the color of blood is related to the oxygen saturation level of hemoglobin. Indeed, as blood deoxygenates, the pinkish skin color (in many individuals) transitions to a bluish hue. This phenomenon allows measurements of the degree of oxygen saturation of blood using, what is commonly referred to as, optical pulse oximetry technology.

Pulse oximetry devices, i.e. oximeters, typically measure and display various blood constituents and blood flow characteristics including, blood oxygen saturation of hemoglobin in arterial blood, the volume of individual blood pulsations supplying the flesh and the rate of blood pulsations corresponding to each heartbeat of the patient. Illustrative are the devices disclosed in U.S. Pat. Nos. 5,193,543, 5,448,991, 4,407,290 and 3,704,706.

As is well known in the art, a pulse oximeter passes light through human or animal body tissue where blood perfuses the tissue, such as a finger or ear, and photoelectrically senses the absorption of light in the tissue. Since oxygenated and deoxygenated hemoglobin absorb visible and near infrared light differently, two lights having discrete frequencies in the range of about 650-670 nm in the red range and about 800-1000 nm in the infrared range are typically passed through the tissue. The amount of transmitted light passed through the tissue varies in accordance with the changing amount of blood constituent, i.e. oxygen (or oxygen saturation), in the tissue and the related light absorption.

Two oxygen saturation parameters can readily be ascertained via oximetry; arterial oxygen saturation and peripheral, arterial oxygen saturation. Arterial oxygen saturation (SaO₂) is based on direct measurement of light absorption in tissue and/or blood based on all commonly measured hemoglobin components. Peripheral, arterial oxygen saturation (SpO₂), as measured by pulse oximetry, is generally determined by measuring the constant (non-pulsatile) and pulsatile light intensities (discussed below) of the two functional components oxyhemoglobin and deoxyhemoglobin, at each of the two noted wavelengths, and correlating the measured intensities to provide peripheral oxygen saturation.

As is also well known in the art, variations in tissue temperature proximate the measurement site can, and in many instances will, affect blood perfusion and, hence, physiological, e.g., oximetry, measurements dependant thereon. Indeed, a rise in tissue temperature induces or triggers a homeostatic reflex, which enhances local blood flow in order to increase the transfer of heat away from the skin. The enhanced blood flow or perfusion will thus enhance the accuracy of an oximetry measurement, since the light transmitted to the tissue will encounter a larger volume of blood.

Various heating means have thus been incorporated in pulse oximeters (and associated systems) to improve blood perfusion adjacent the sensor. Illustrative are the oximetry sensors disclosed in U.S. Pat. Nos. 4,926,867, 5,299,570, 4,890,619 and 5,131,391.

In U.S. Pat. No. 4,926,867, an oximetry sensor is disclosed that includes a metal plate that functions as a heater. According to the invention, the heater is adapted and positioned to heat the tissue proximate the sensor to enhance blood perfusion. A separate thermistor is also provided to monitor the amount of heat transmitted to the tissue by the heater.

U.S. Pat. Nos. 5,299,570 and 4,890,619 disclose oximetry sensors that employ ultrasonic energy to enhance blood perfusion. The blood perfusion is similarly enhanced primarily proximate the sensor.

Various substances have also been applied to the skin (or tissue site) to enhance blood perfusion. Illustrative are the pulse oximeter methods disclosed in U.S. Pat. Nos. 5,392,777, 5,267,563 and 6,285,896.

In U.S. Pat. Nos. 5,392,777 and 5,267,563, a counterirritant is applied to the skin prior to attachment of the oximetry sensor. In U.S. Pat. No. 6,285,896, a vasodilating substance is applied to the skin prior to attachment of the oximetry sensor to reduce the effects of localized oxygen consumption and to increase blood fraction.

Although the noted sensor systems and methods provide effective means to enhance blood perfusion, there are a number of disadvantages and drawbacks associated with the systems and methods. A major drawback is that the enhanced blood perfusion realized by the conventional sensor systems and methods is typically localized, i.e. proximate the sensor. As discussed in detail herein, applicants have found that the signal-to-noise ratio of a physiological sensor; particularly, an oximetry sensor (and, hence, the accuracy of any physiological characteristic, e.g., O₂ saturation, determined therefrom) can be significantly enhanced by heating an entire organ or appendage, e.g., ear or hand, prior to or in conjunction with measuring and/or determining a physiological characteristic, such as O₂ saturation.

Another major drawback is that conventional sensor systems and methods that employ heating means do not include any means of regulating the heating means, i.e. heating profile, based on the body's response to the applied heat, i.e. heat stimuli.

A further drawback associated with conventional sensor systems and methods that employ heating means is that they are typically limited to one physiological sensor, i.e. an oximetry sensor.

A further drawback is that virtually all of the conventional sensor heating means comprise means for heating the sensor (or housing thereof) or a member that is integral thereto, e.g., heated plate. Such heating means necessitates frequent site changes to avoid thermal injury, which makes the monitoring method (employing the heating means) more labor intensive and costly than other non-invasive monitoring methods.

It would therefore be desirable to provide an integrated sensor apparatus and system that substantially reduces or overcomes the disadvantages and drawbacks associated with conventional sensor methods and systems, such as pulse oximeter sensor methods and systems.

It is therefore an object of the invention to provide an integrated sensor apparatus and system that substantially reduces or overcomes the disadvantages and drawbacks associated with conventional sensor methods and systems.

It is another object of the invention to provide an integrated sensor apparatus and system, and method based thereon, that enhance the accuracy of physiological measurements and determinations made therefrom.

It is another object of the invention to provide an integrated sensor apparatus and system that includes at least one pulse oximeter sensor and heating means to enhance blood perfusion in one or more body sites proximate positioned pulse oximeter sensors.

It is another object of the invention to provide an integrated sensor apparatus and system that includes heating means that is adapted to heat at least one significantly larger tissue region, such as an entire ear and/or hand, prior to or in conjunction with obtaining a physiological reading therein.

It is another object of the invention to provide an integrated sensor apparatus and system that includes multiple physiological sensors and associated heating means that are adapted to selectively heat one or more tissue regions proximate positioned physiological sensors.

It is another object of the invention to provide an integrated sensor apparatus and system that includes one or more algorithms that are designed and adapted to regulate the heating means based on the body's response to the applied heat, i.e. heat stimuli.

It is another object of the invention to provide an integrated sensor apparatus and system that includes means for applying and regulating the applied force (or pressure) to a tissue site that is subject to the heat stimuli.

It is yet another object of the invention to provide an integrated sensor apparatus and system that includes multiple physiological sensors to determine multiple physiological characteristics, such as arterial oxygen saturation and peripheral, arterial oxygen saturation, blood pressure, and electrical signals and/or impulses associated with heart function.

### SUMMARY OF THE INVENTION

In accordance with the above objects and those that will be mentioned and will become apparent below, in one embodiment of the invention, there is provided an integrated physiological sensor system, comprising (i) a plurality of physiological sensors, the plurality of physiological sensors including at least a first physiological sensor adapted to measure pulse amplitude at a target measurement site on a subject's body and a second physiological sensor adapted to monitor electrical impulses associated with the subject's heart function, and (ii) means for heating a tissue region on the subject's body, whereby blood perfusion of the tissue region is enhanced, the tissue region including the target measurement site and extending beyond the target measurement site.

In one embodiment of the invention, the system includes a third sensor adapted to monitor blood pressure.

In accordance with another embodiment of the invention there is provided an integrated physiological sensor system, comprising: (i) a plurality of physiological sensors, the plurality of physiological sensors including at least a first physiological sensor adapted to measure pulse amplitude at a first target measurement site on a subject's body and a second physiological sensor adapted to monitor electrical impulses associated with the subject's heart function, (ii) means for heating a tissue region on the subject's body, whereby blood perfusion of the tissue region is enhanced, the tissue region including the first target measurement site and extending beyond the first target measurement site, (iii) a first heat sensor adapted to monitor skin surface temperature of the tissue region, and (iv) a processor in communication with the heating means and the first heat sensor, the processor including at least one algorithm for regulating the heating means based on a physiological response of the subject's body to heating of the tissue region.

In one embodiment of the invention, the first physiological sensor comprises a pulse oximetry sensor having a signal-to noise ratio.

In one embodiment of the invention, the processor includes stored pulse amplitude and skin surface temperature data, and wherein the algorithm is adapted to compare first pulse amplitude measured by the first physiological sensor and first skin surface temperature measured by the first heat sensor to the stored amplitude and skin surface temperature data, and adjust the heat provided by the heating means base on the comparison, whereby the signal-to-noise ratio of the first physiological sensor is optimized.

In one embodiment of the invention, the system includes a third physiological sensor adapted to measure pulse amplitude at a second target measurement site that is close to the first target measurement site, but independent thereof, and a second heat sensor adapted to monitor skin surface temperature of the second target measurement site, the third physiological sensor and the second heat sensor being in communication with the processor.

In one embodiment of the invention, the algorithm is adapted to compare first pulse amplitude measured by the first physiological sensor and first skin surface temperature measured by the first heat sensor to second pulse amplitude measured by the third physiological sensor and second skin temperature measured by the second heat sensor, and adjust the heat provided by the heating means base on the comparison, whereby the signal-to-noise ratio of the first physiological sensor is optimized.

In one embodiment of the invention, at least the first physiological sensor includes at least one lead operatively connected to the first physiological sensor and the processor to facilitate communication by and between the first physiological sensor and the processor, the first physiological sensor lead including quick-disconnect means, and the processor further includes a disconnect algorithm that is adapted to monitor the quick-disconnect means and limit operation of the system after a predetermined number of subsequent re-connections of the quick-disconnect means after a predetermined period of time.

In one embodiment of the invention, the system includes at least one ear adapter adapted to engage an ear of the subject, the ear adapter including the first physiological sensor.

In one embodiment of the invention, the ear adapter further includes means for applying pressure to the ear lobe of the engaged ear and at least one pressure sensor adapted to monitor the applied pressure on the ear lobe, and the processor further includes an ear pressure algorithm adapted to regulate the applied pressure on the ear lobe.

In accordance with another embodiment of the invention, there is provided a method for determining a physiological characteristic, comprising the steps of (i) providing at least a first physiological sensor that is adapted to measure pulse amplitude at a first tissue region on a subject's body, the first physiological sensor having a signal-to-noise ratio, (ii) disposing the first physiological sensor proximate the first tissue region, (iii) heating the first tissue region to an interrogation temperature, (iv) measuring a first pulse amplitude at the first tissue region with the first physiological sensor during heating of the first tissue region, (v) measuring a first temperature of the first tissue region during heating of the first tissue region, (vi) providing demographic pulse amplitude and skin surface temperature data, and (vii) providing a temperature algorithm that is adapted to adjust the interrogation temperature as a function of the first pulse amplitude and the first tissue region temperature and the demographic pulse amplitude and skin surface temperature data, whereby the signal-to-noise ratio of the first physiological sensor is optimized.

In accordance with another embodiment of the invention, there is provided a method for determining a physiological characteristic, comprising the steps of (i) providing a first physiological sensor that is adapted to measure pulse amplitude at a first tissue region on a subject's body, the first physiological sensor having a signal-to-noise ratio, (ii) disposing the first physiological sensor proximate the first tissue region, (iii) providing a second physiological sensor that is adapted to measure pulse amplitude at a second tissue region on the subject's body, the second tissue region being close to, but independent of the first tissue region, (iv) disposing the second physiological sensor proximate the second tissue region, (v) heating the first tissue region to an interrogation temperature, (vi) measuring a first temperature of the first tissue region during heating of the first tissue region, (vii) measuring a first pulse amplitude at the first tissue region with the first physiological sensor during heating of the first tissue region, (viii) measuring a second temperature of a second tissue region, (ix) measuring a second pulse amplitude at the second tissue region with the second physiological sensor, and (x) providing an algorithm that is adapted to adjust the interrogation temperature as a function of the first and second tissue region temperatures and the first and second pulse amplitudes, whereby the signal-to-noise ratio of the first physiological sensor is optimized.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a schematic illustration of a conventional pulse oximeter system;

FIGS. 2 and 3 are schematic illustrations of one embodiment of an integrated physiological sensor system, according to the invention;

FIG. 4 is a flow chart of one embodiment of a temperature algorithm, according to the invention;

FIG. 5 is a schematic illustration of the integrated sensor system shown in FIG. 3, showing heat applied to an ear of a subject and measurement of light absorption of (i.e. oximeter reading) the subject's heated ear, according to the invention;

FIGS. 6-10 are illustrations of one embodiment of an ear adaptor (i.e. central circulation sensor system), according to the invention;

FIG. 11 is an illustration of another embodiment of an ear clip, according to the invention;

FIG. 12 is a schematic illustration of the integrated sensor system shown in FIG. 3, showing heat applied to an appendage, i.e. arm and/or hand, of a subject and measurement of light absorption of the subject's heated finger, according to the invention;

FIGS. 13 and 14 are illustrations of one embodiment of a hand warmer/finger adapter (i.e. peripheral circulation system), according to the invention;

FIG. 15 is an illustration of another embodiment of a finger adapter, according to the invention;

FIGS. 16 and 17 are schematic illustrations of another embodiment of an integrated sensor system having a plurality of sensors and associated heating means, according to the invention;

FIG. 18 is a schematic illustration of the integrated sensor system shown in FIG. 17, showing heat applied to an ear and arm of a subject and measurement of light absorption of the subject's heated ear and finger, according to the invention;

FIG. 19 is an illustration of an IR portion of an oximetry plethysmogram obtained on an area of a subject's ear at a baseline temperature in the range of approximately 29-32° C., according to the invention;

FIGS. 20 and 21 are illustrations of IR portions of oximetry plethysmograms obtained on an area of the ear of first and second subjects, respectively, at an elevated temperature in the range of approximately 35-37° C., according to the invention; and

FIGS. 22 and 23 are graphical illustrations showing the effect of different heating method or conditions on pulse amplitude for subjects ranging in age from 71-94 years of age and 25-55 years of age, respectively, according to the invention.

### DETAILED DESCRIPTION OF THE INVENTION

Before describing the present invention in detail, it is to be understood that this invention is not limited to particularly exemplified methods or systems as such may, of course, vary. Thus, although a number of methods and systems similar or equivalent to those described herein can be used in the practice of the present invention, the preferred methods and systems are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise.

### Definitions

The term “physiological sensor”, as used herein, means and includes any sensor that is adapted to communicate with the body and sense or measure a physiological parameter or characteristic, such as S_{P}0₂, blood pressure, body temperature, etc.

The terms “pulse oximetry sensor”, “pulse oximeter”, “oximetry sensor” and “oximeter”, as used herein, mean and include any conventional light-reflecting oximeter or sensor that is adapted to sense or measure light absorption in tissue and/or blood.

The term “oximeter reading”, as used herein, means and includes a measure of light absorption in tissue and/or blood.

The term “heating means”, as used herein, means and includes any means of increasing the core or tissue temperature of a subject, including, without limitation, one or more (i.e. a combination of) devices that transmit heat energy, such as thermoelectric heating devices (e.g., heating elements of various sizes, shapes, materials, etc. that are adapted to cooperate with various heating apparatus and/or configurations, such as a heated glove), contact heaters, lamps, heating blankets, etc., heated rooms, heated liquids, devices that transmit ultrasonic or photoelectric energy, and mentholated, counterirritant and/or vasodilating substances. The term “heating means” also means and includes passive heating means, i.e. means for limiting heat from escaping a specific tissue region of the body.

The terms “patient” and “subject”, as used herein, is meant to mean and include humans and animals.

The present invention substantially reduces or eliminates the disadvantages and drawbacks associated with conventional physiological sensor apparatus and sensors. In one embodiment of the invention, the integrated physiological sensor apparatus and system includes a pulse oximeter sensor and associated heating means that is adapted to heat a small and/or large tissue region or site, such as an entire organ or appendage, prior to or in conjunction with obtaining an oximeter reading. In another embodiment, the integrated physiological sensor apparatus and system includes a plurality of pulse oximeter sensors and associated heating means that are similarly adapted to selectively heat small and/or large tissue regions or sites prior to or in conjunction with obtaining oximeter readings.

In some embodiments of the invention, the integrated physiological apparatus and systems include at least one additional physiological sensor that is adapted to monitor a further physiological characteristic or parameter, such as blood pressure, tissue temperature, and electrical signals and/or impulses associated with heart function.

As discussed in detail below, Applicants have found that the signal-to-noise ratio of a physiological sensor, such as an oximetry sensor, (and, hence, the accuracy of any physiological characteristic, e.g., O₂ saturation, determined therefrom) can be significantly enhanced by heating a significantly larger tissue region, i.e. a region that extends beyond the target measurement site and/or region in direct communication with the sensor, prior to or in conjunction with obtaining a physiological measurement, e.g., O₂ saturation, at the target measurement site.

Applicants have also recognized that the body's response to applied heat, i.e. heat stimuli, can, and in most instances will, vary from patient-to-patient. Thus, in a preferred embodiment of the invention, the integrated physiological sensor apparatus and systems include one or more algorithms that are adapted to regulate the heating means based on a body's response to the heat stimuli.

Although the integrated sensor apparatus, systems and methods of the invention are primarily described herein in conjunction with pulse oximetry sensors and systems, and measurements (or readings) obtained therewith, it is understood that the sensor apparatus, systems and methods are not limited to pulse oximetry and determinations made therefrom. Indeed, in some embodiments of the invention, the integrated physiological apparatus and systems include at least one additional physiological sensor, which is adapted to monitor and/or determine a physiological characteristic based on the wave form, or amplitude or shape of a plethysmogram. In further envisioned embodiments of the invention, the integrated physiological apparatus and systems include at least one physiological sensor that is adapted to monitor blood pressure.

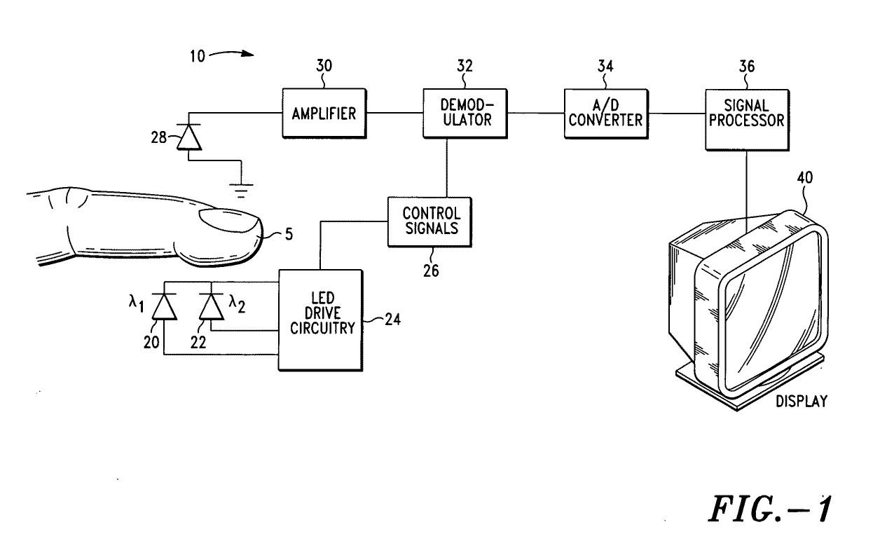

Referring now to FIG. 1, there is shown one embodiment of a conventional oximetry sensor and associated system (denoted generally “10”) that can be employed within the scope of the present invention. As illustrated in FIG. 1, the oximetry sensor 10 preferably includes two emitters 20, 22 and detector 28, which are positioned adjacent the tissue being analyzed, i.e. finger 5.

Two lights are emitted by the emitters 20, 22; in one embodiment, a first light having a discrete wavelength in the range of approximately 650-670 nanometers in the red range and a second light having a discrete wavelength in the range of approximately 800-950 nanometers. The lights, in the illustrated embodiment, are transmitted through finger 10 via emitters 20, 22 and detected by detector 28.

The emitters 20, 22 are driven by drive circuitry 24, which is, in turn, governed by control signal circuitry 26. Detector 28 is in communication with or connected to amplifier 30. The signal from amplifier 30 is transmitted to demodulator 32, which is also synchronized to control signal circuitry 24. The demodulator 32, which is employed in most pulse oximeter systems, removes any common mode signals present and splits the time multiplexed signal into two (2) channels, one representing the red voltage (or optical) signal and the other representing the infrared voltage (or optical) signal.

The signal from the demodulator 32 is transmitted to an analog-digital converter 34. As is well known in the art, the output signal from the demodulator 34 is typically a time multiplexed signal comprising (i) a background signal, (ii) the red light range signal, and (iii) the infrared light range signal.

The desired computations are performed on the output from the converter 34 by signal processor 36 and the results transmitted to and displayed by display 40.

Referring now to FIG. 2, there is shown a schematic illustration of one embodiment of an integrated physiological sensor apparatus and system of the invention (denoted generally “100”). As illustrated in FIG. 2, the system 100 includes oximetry sensor 10 (discussed above), heating means 40 and, optionally, at least one monitor or display 55.

As will readily be appreciated by one having ordinary skill in the art, various oximetry sensors (and systems) can be employed within the scope of the invention. Thus, although the sensor systems 100, 200, discussed in detail below, employ oximetry sensor 10 (shown in FIG. 1), such use and discussion herein should not be deemed limiting.

Referring back to FIG. 2, in some embodiments of the invention, the heating means 40 is connected to or in communication with, e.g., wireless communication, with the oximetry sensor 10. Similarly, in some embodiments, heating means 40 is in communication with the display 50, whereby the heat transmitted by the heating means 40 can be displayed and, hence, monitored.

In some embodiments of the invention, the heating means 40 includes heat regulating means (shown in phantom and designated “46”), e.g., heating blanket, or integral control means, that is adapted to monitor and regulate the heat transmitted by the heating means 40.

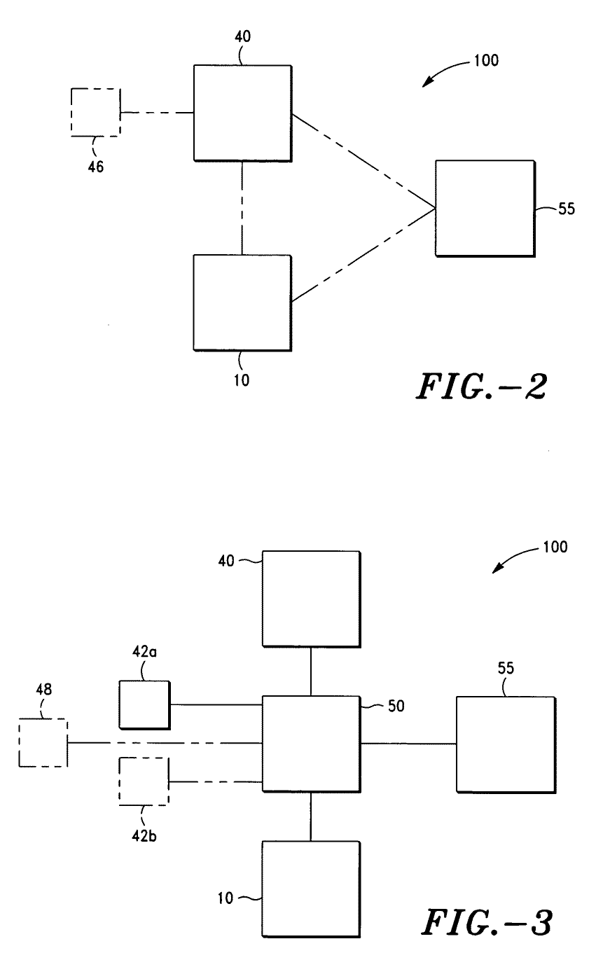

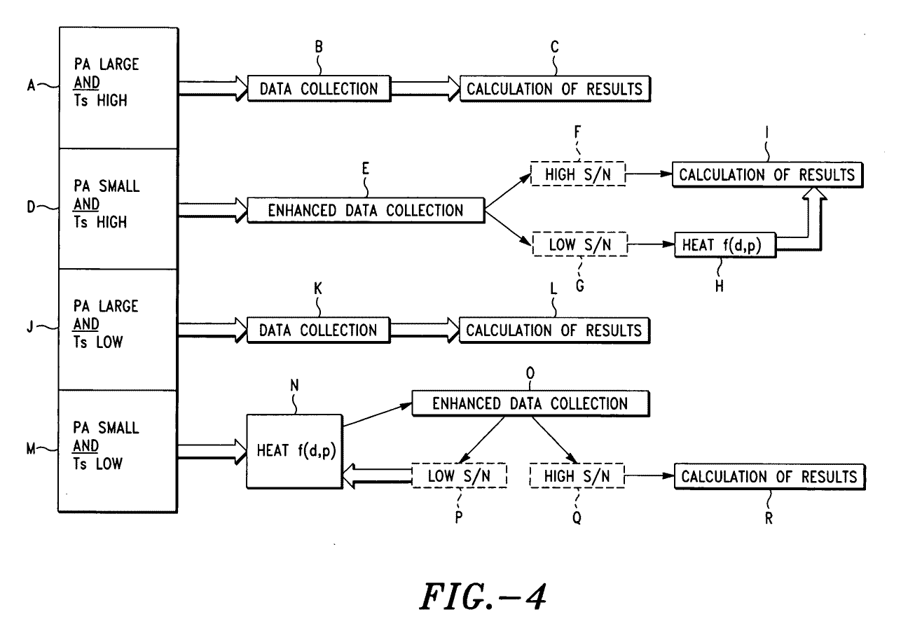

Referring now to FIG. 3, in a preferred embodiment of the invention, system 100 further includes at least one heat sensor 42a that is adapted to be disposed proximate the tissue region being heated by the heating means 40 and monitor the temperature of the heated tissue region, and processor means (or processor) 50 that is in communication with heating means 40, oximetry sensor 10, heat sensor 42a and display 55. The processor means 50 is preferably programmed and adapted to regulate heating means 40 and/or oximetry sensor 10 and/or the output displayed on display 55.

As illustrated in FIG. 3, in some embodiments of the invention, the system 100 additionally includes at least a second heat sensor (shown in phantom and designated “42b”) that is adapted to be disposed at a target body site or region that is close to, but independent from, the heated tissue region to monitor the temperature of the target region and, hence, the body's response to the heat stimuli. The second heat sensor 42b is preferably similarly in communication with processor means 50, which, as discussed in detail below, includes at least one algorithm that is designed and adapted to regulate the heating means 40 based on the body's response to the heat stimuli.

In further embodiments of the invention, the system 100 further includes an ECG sensor (shown in phantom and designated “48”) that is adapted to monitor electrical signals and/or impulses associated with heart function. In the noted embodiments, the ECG sensor 48 would similarly be in communication with processor means 50.

In the illustrated embodiment, the ECG sensor 48 comprises a separate, stand-alone sensor (and associated system). However, in a further envisioned embodiment of the invention (not shown), the ECG sensor is incorporated in one or more oximetry sensors, e.g., oximetry sensor 10 and/or sensors 10a, 10b, discussed below. The integrated system would thus eliminate the pads and wires typically associated with conventional ECG sensors. The resulting vector, which would be determined by position of the oximetry sensor(s), would provide good S/N.

In additional (envisioned) embodiments, the system includes at least one additional physiological sensor (not shown) that is adapted to monitor a further physiological characteristic or parameter, such as blood pressure.

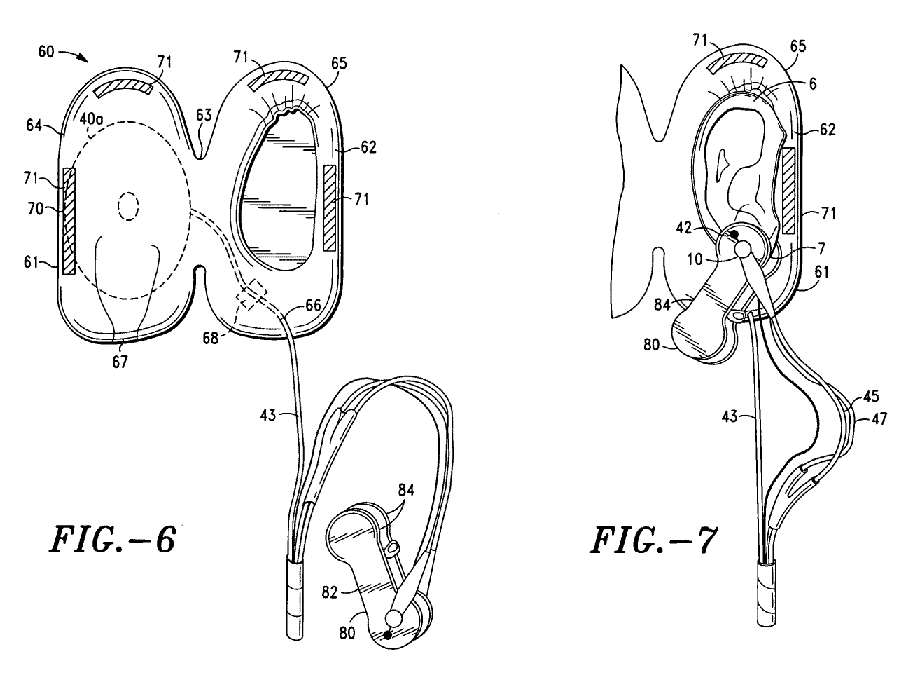

As indicated above, in one embodiment of the invention, the heating means 40 of the invention is adapted to transmit heat energy to a large or extended tissue region, i.e. a tissue region that extends beyond the target measurement site and/or the tissue region that is proximate to or in direct communication with the sensor (see, e.g., FIGS. 5 and 12), prior to or in conjunction with obtaining an oximeter or other physiological reading. In some embodiments of the invention, the heating means 40 is also adapted to heat a smaller tissue region, preferably, a tissue region proximate a physiological sensor, e.g., oximetry sensor 10.

The heating means 40 of the invention can thus comprise any means of increasing the core or tissue (or skin) temperature of a subject, including, without limitation, devices that transmit heat energy, such as thermoelectric heating devices (e.g., heating elements of various sizes, shapes, materials, etc. that are adapted to cooperate with various heating apparatus and/or configurations, such as a heated glove), contact heaters, lamps, heating blankets, etc., heated rooms, heated liquids, devices that transmit ultrasonic or photoelectric energy, and mentholated, counterirritant and/or vasodilating substances, and passive heating means, i.e. means for limiting heat from escaping a specific tissue region of the body. As indicated above, the heating means 40 can also comprise two or more of the noted devices and means, e.g. two heat lamps.

According to the invention, in addition to the heat profile effectuated by a temperature algorithm of the invention (discussed in detail below), the heat or heat energy provided by the heating means 40 can be substantially steady state (or constant) or varied, e.g. oscillated or any function of time-varied heating.

According to the invention, the heat or heat energy transmitted by the heating means 40 and applied to the tissue is sufficient to induce or trigger an optimal homeostatic reflex, whereby tissue perfusion of the heated tissue region is enhanced, without burning the patient. As will be appreciated my one having ordinary skill in the art, the amount of heat or heat energy that would be necessary to trigger an optimal homeostatic reflex will vary from patient-to-patient, site-to-site on the same patient, as well as over time, depending on physical and/or mental health condition, metabolic status, exertion or fatigue and prior thermal conditioning or exposure.

Applicants have, however, found that when the skin of a patient is heated up to a generally tolerable temperature range of approximately 40-42° C., arterioles in the blood vessel network that spread in the shallow layer within the dermis respond to the heat stimulus by active expansion of the inner diameters of the arterioles and general vasodialation. The expanded diameter results in a lowered resistance to blood flow and, hence, increased blood flow therethrough. Thus, in one embodiment of the invention, to optimize the increase of perfusion, the skin or tissue of the patient is initially heated to at least a temperature of approximately 35° C. or, at a minimum, 3° C. above the skin or surface temperature and below a temperature of approximately 42° C. to avoid burning the patient.

A key feature and advantage of the integrated physiological sensor apparatus and systems of the invention is the capability of applying heat or heat energy over a large tissue region, such as an entire organ or appendage, prior to or in conjunction with taking an oximeter (or other physiological) reading. As indicated above, Applicants have found that the signal-to-noise ratio of a physiological sensor; particularly, an oximetry sensor (and, hence, the accuracy of any physiological characteristic, e.g., O₂ saturation, determined therefrom) can be significantly enhanced by heating a large tissue region prior to or in conjunction with obtaining an oximeter reading. Indeed, Applicants have realized about one order of magnitude improvement in the signal-to-noise ratio by virtue of the systems and means of the invention.

As will readily be appreciated by one having ordinary skill in the art, an order of magnitude increase in blood perfusion is significant in that the resulting signal strength enables physiological measurement(s) at an optimum site, such as a site proximate the central circulation, which is, by design, much less affected by vasoconstriction and, which is more proximal the heart and aorta. Such sites were heretofore deemed inaccessible and there was insufficient sensor signal strength to yield useful and high quality measurements, i.e. a quality that is comparable to conventional sites when non-constricted, such as the finger.

According to the invention, the large tissue region that is subjected to heating can, of course, comprise the entire body of the patient. The heating means 40, in this instance, could thus comprise a heated liquid bath or a heated room, such as a sauna.

More preferably, the larger tissue region comprises an entire organ or appendage and, in some embodiments, the adjoining tissue structure.

As will also be appreciated by one having ordinary skill in the art, the body's response to the heat stimuli, e.g. homeostatic reflex, will also vary from patient-to-patient. Thus, as indicated above, in some embodiments of the invention, the processor means 50 includes at least one algorithm that is adapted to regulate the heating means 40 (and/or physiological sensors, e.g. oximetry sensor 10) based on the body's response to the heat stimuli (hereinafter referred to as “temperature algorithm”).

In one embodiment of the invention, the temperature algorithm is adapted to compare measured pulse amplitude and skin surface temperatures at a first site (or region) within the heated tissue region to measured pulse amplitude and skin surface temperature at a second site that is close to the first site, but independent thereof, and adjust the applied heat based on the comparison to optimize the signal-to-noise ratio. By way of example, at a first tissue region a low measured pulse amplitude of 1% is measured at a skin surface temperature of 28° C., whereas the pulse amplitude at a second tissue region that is close to, but independent of the first tissue region is an acceptable 5% at a temperature of 35° C. In this case, the temperature algorithm would adjust, i.e. slightly raise, the temperature at the first tissue region several degrees.

In some embodiments of the invention, the temperature algorithm is further adapted to determine (or estimate) at least one core temperature as a function of the measured temperature at the second tissue region and adjust the applied heat in response thereof.

In another embodiment of the invention, the temperature algorithm is adapted to optimize the signal-to-noise ratio by comparing measured pulse amplitude and skin surface temperature at a first site (or region) within the heated tissue region to stored demographic data, i.e. pulse amplitude and skin surface temperature data from a plurality of subjects, and adjust the applied heat based on the comparison. Preferably, the stored demographic data includes measured pulse amplitudes and skin surface temperatures of a second tissue region on a plurality of subjects during heating of a first tissue region on the second subjects' body, the second tissue region being close to, but independent of the first tissue region.

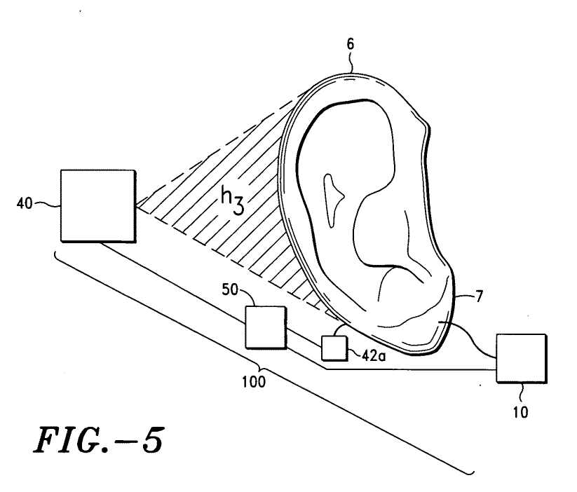

Referring to FIG. 4, there is shown a schematic illustration (or flow chart) reflecting the noted temperature algorithm. As illustrated in FIG. 4, there are a total of four combinations of pulse amplitude and skin temperature (designated A, D, J, and M), reflecting the demographic data collection parameters. In one embodiment of the invention, pulse amplitude (PA) is determined from the IR channel of the plethysmogram or oximetry probe based on the ratio of the measured difference in detector output between the maximal systole and the minimal diastole divided by the total IR intensity, in percent, wherein large pulse amplitude is defined as >0.5%, preferably, >2.0%.

In one embodiment, a high skin temperature (Ts) is defined as >20° C., more preferably, >30° C., even more preferably, >36° C., and a low skin temperature is defined as <20° C., more preferably, <30° C., even more preferably, <36° C.

In one embodiment of the invention, the signal-to-noise ratio (S/N) is based on the ratio of pulse amplitude divided by the uncertainty of the signal, wherein a high signal-to-noise ratio is defined as >5, more preferably, >30, and a low signal-to-noise ratio is defined as <5, more preferably, <30.

As illustrated in FIG. 4, in case A, the signal quality is high during data collection and results calculated therefrom. In case B, there is a bifurcation after extended or enhanced data collection (E), in that the quality of the S/N is either sufficient (F), in which case the results can be calculated, or the SIN quality is not sufficiently good (G), in which case heat is applied to the body site in a variety of ways, e.g., constant, at different heating rates or intermittently, until the quality of the S/N is sufficient to proceed with calculation of results (I).

In case J, there is an unusual physiological combination that results in high perfusion and good S/N to warrant proceeding to results (L). In case M, there is a mandatory application of heat to bring tissue perfusion up a level that enables data collection and calculation of results (R). According to the invention, the heat treatment and decision making can be automated in iterative ways (P,N,O . . . P,N,Q,R)

Referring now to FIG. 5, there is shown a schematic illustration of the application of heat to an entire ear 6 by heating means 40 (shown as heat zone “h₃”). According to the invention, the heat applied to the ear 6 can be applied in such a manner (e.g., intensity and/or direction) that only a portion of the ear 6 is heated or the entire ear 6 is heated or the entire ear 6 and the adjoining tissue region or tissue and/or bone structure of the head are heated (unless otherwise stated, referred to collectively herein as “heated ear”).

Thus, in one embodiment of the invention, a significant portion of the ear 6, more preferably, the entire ear 6 is heated. In another embodiment, the entire ear 6 and the adjoining tissue region or tissue and/or bone structure of the head (referred to collectively hereinafter as “adjoining tissue region) are heated.

According to the invention, the heat can be applied to the ear 6 (or the entire ear 6 and the adjoining tissue region) prior to or in conjunction with obtaining an oximeter (and/or other physiological) reading, on a site therein, preferably, the ear lobe 7.

As indicated, according to the invention, various heating means can be employed within the scope of the invention to heat a desired portion of the ear 6 or the entire ear 6. Various means for retaining and positioning the heating means 40 and sensors, e.g., oximetry sensor 10 and heat sensor 42a, can similarly be employed with the scope of the invention.

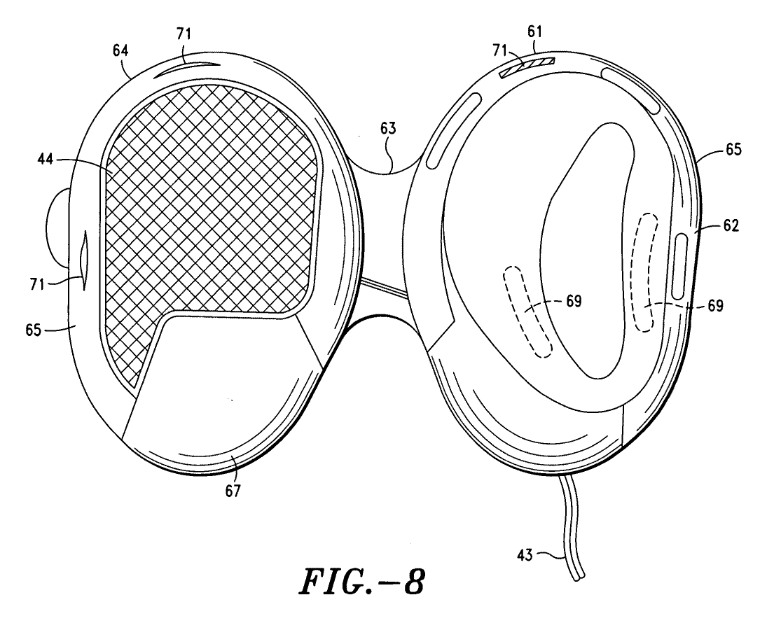

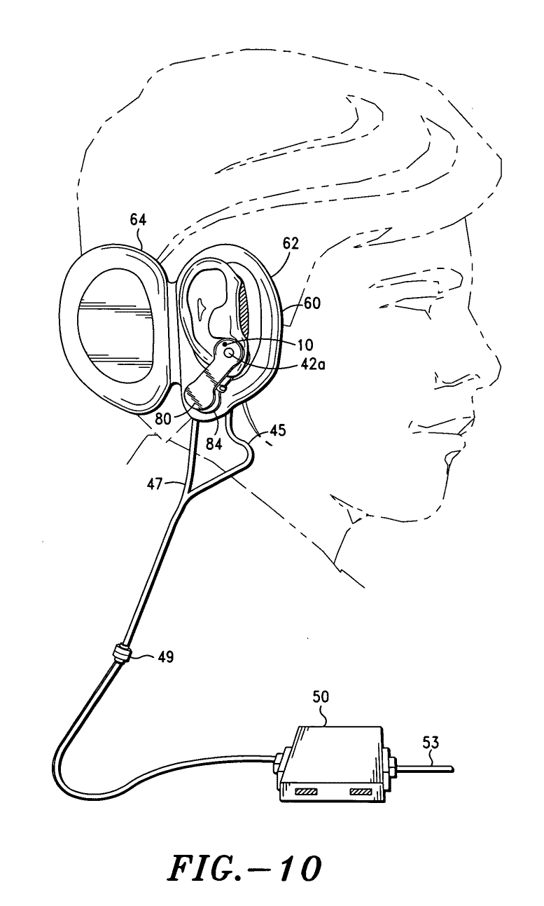

Referring now to FIGS. 6-10, there is shown one embodiment of an ear adapter 60 that includes heating means, multiple sensors and means for retaining and positioning same (i.e. central circulation system). As illustrated in FIGS. 6 and 7, the ear adapter 60 comprises a clam shell shaped housing 61 having a base 62 that is adapted to encase the outer portion of the ear 6, a middle hinge region 63, and a cover 64 (having a similar shape and configuration as the base 62) that is adapted to be folded onto the base 62 via hinge region 63 (see FIG. 9).

According to the invention, the ear adapter housing 61 can be constructed of various materials. Preferably, the housing 61 is constructed of materials having minimum weight, provide maximal patient comfort, enable efficient heat control and effective heat distribution within the adapter 60, and allow for moisture (e.g. perspiration) to dissipate and, hence, prevent condensation within the adapter 60.

In one embodiment of the invention, the ear adapter housing 61 includes an optional protective outer layer that is preferably constructed of ABS or like material and includes a raised closed cell polyethylene foam, e.g., TM3201 Medical Foam, distributed by MACtac, that is disposed proximate the perimeter 65 of the base 62 and cover 64.

As further illustrated in FIGS. 6 and 7, the ear adapter housing 61 includes at least one access portal 66 that is preferably disposed in the cover 64 and is adapted to receive the heating means leads 43, and a recessed region 67 that is adapted to receive and position the ear clip 80. In the noted embodiment, the housing 61 also includes an internal anchor 68 that provides strain relief for the heating means leads 43.

The ear adapter housing 61 further includes means for positioning and retaining the cover 64 to the base 62, and means for positioning and retaining the base 62 on the ear 6.

According to the invention, various means can be employed within the scope of the invention to position and retain the cover 64 to the base 62, such as snap and Velcro retention systems. In the illustrated embodiment, a Velcro system 70 is employed to position and retain the cover 64 to the base 62. As illustrated in FIG. 6, the Velcro system 70 includes at least one, preferably, a plurality of Velcro strips 71 that are disposed proximate the outer perimeter 65 of the cover 64 and base 62.

Various means can similarly be employed to position and retain the base 62 to the ear 6, such as surgical tape, biocompatible adhesives, etc. In one embodiment of the invention, a hydrogel, e.g. medical grade hydrogel tape, distributed by M&C Specialties Co., is employed to retain the base 62 to the ear 6.

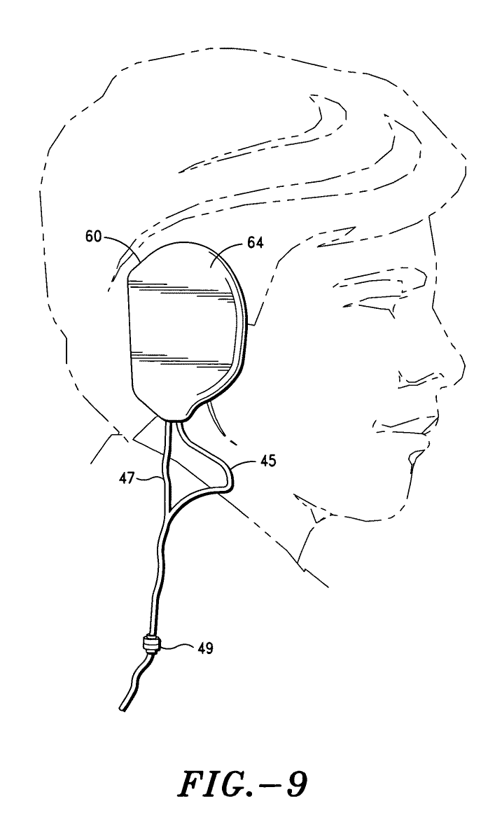

Referring to FIG. 8, in the noted embodiment, the base 62 includes at least one, preferably, a plurality of pockets 69 that are adapted to receive a hydrogel.

As indicated above, the ear adapter 60 includes heating means and, preferably, multiple sensors. According to the invention, the heating means can comprise any of the aforementioned heating means, such as radiative, convective and conductive heating means.

In the illustrated embodiment, the heating means (designated “40a”) preferably comprises a flex circuit heater, such as a Minco PDF Heater. As illustrated in FIG. 6, the heating means 40a is disposed within the adapter housing 61 in cover 64.

In a preferred embodiment, the cover 64 includes a heating means region 44 that is adapted to enhance the heat flow from the heating means 40a. According to the invention, various means and materials can be employed to enhance the heat flow from the heating means 40a. In one embodiment of the invention, which is illustrated in FIG. 8, the heating means region 44 is constructed of cross-linked polyethylene to enhance the uniformity of heat flow from the heating means 40a, as well as to prevent local hot spots in direct contact with the skin.

Referring back to FIGS. 6 and 7, the ear adapter 60 includes an ear clip 80 that is adapted to receive at least one optical interrogating means, such as oximetry sensor 10, and at least one tissue temperature sensing means, such as temperature sensor 42a, and position the optical interrogating means and tissue temperature sensing means on the ear 6.

According to the invention, the ear clip 80 can comprise various configurations and materials. In the illustrated embodiment, the ear clip 80 includes two hingedly connected elongated sections 82, 84 that are designed to receive an ear lobe 7 therebetween. In one embodiment, the clip sections 82, 84 are preferably constructed of a thermoplastic elastomer, such as ABS or like material

Preferably, the ear clip 80 provides an ear lobe engagement force no less than approximately 5 mm Hg and no greater than 50 mm Hg. More preferably, the ear clip 80 provides an ear lobe engagement force no less than 10 mm Hg and no greater than 25 mm Hg; primarily, to minimize venous effects, e.g., optical effects due to venous pooling or venous pulsation, as caused indirectly by arterial pulsation. As is well known in the art, the elimination of any venous component enhances the accuracy of the arterial measurement of physiological parameters.

According to the invention, various tissue temperature sensing means, i.e. temperature sensor 42a, can be employed within the scope of the invention, including non-contact thermometer-type infrared radiation devices and contact thermo-sensor devices, e.g., thermocouples. In a preferred embodiment, the temperature sensor 42a comprises a thermocouple-type skin contacting device that protrudes approximately 0.5 mm to from the sensor surface to ensure good skin contact.

In a preferred embodiment of the invention, the temperature sensor 42a is disposed proximate or alternatively contra-lateral to the tissue optical interrogation region, whereby the temperature reading is not affected by the heating means (and heat provided therefrom) and is substantially representative of the tissue core temperature. A preferred location on the ear lobe 7 is thus proximate the skin surface on the opposite side of the ear lobe 7 relative to the heating means 40a, and, hence heated ear lobe 7 region.

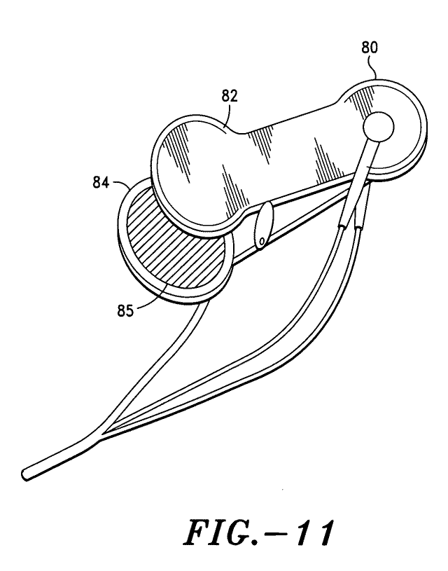

Referring to FIG. 10, the ear adapter 60 further includes processor means 50 that is operatively connected to the temperature sensor 42a and oximetry sensor 10 via leads 45, 47, respectively. As illustrated in FIG. 10, the ear adapter 60 additionally includes quick-disconnect means 49 that is adapted to effectuate disconnection and connection of leads 45, 47.

As indicated, the processor means 50 includes at least one of the aforementioned ear temperature algorithms. In some embodiments the processor means 50 further includes a disconnect algorithm that is adapted to monitor the quick-disconnect means 49 and, hence, connection, disconnection and use, i.e. operating intervals, of the ear adapter 60. By way of example, in a preferred mode of operation, the ear adapter 60 is limited to a single use on one patient. The disconnect algorithm would thus allow full operation capability after the initial connection of leads 45, 47 to the processor means via quick-disconnect means 49 and prohibit operation on a subsequent connection (or up to a predetermined number of re-connections, e.g., 3) after a predetermined period of time (e.g., 10-20 min.). The disconnect algorithm would thus facilitate “temporary” disconnection of the ear adapter 60 from the processor means 50 to, for example, move or bath the patient, adjust the adapter 60, etc.

In some embodiments of the invention, the processor means further includes at least one ear or ear lobe pressure algorithm (discussed below), one or more signal acquisition, digitization and processing algorithms, such as disclosed in U.S. Pat. Nos. 7,184,809 and 7,251,987, and U.S. application Ser. Nos. 11/418,937 and 11/901,985, and one or more physiological algorithms for determining cardiac characteristics/functions, such as disclosed in U.S. application Ser. Nos. 11/700,328, 11/881,103 and 12/011,122.

According to the invention, the processor means 50 is in communication with a monitor (not shown) that preferably includes display 55. The noted communication can be achieved via a lead wire 53 or wirelessly. Such wireless connection can comprise a Bluetooth or similar means of wireless communication.

In some embodiments of the invention, the monitor also include processor means, which can include the ear temperature and disconnect algorithms discussed above (and pressure algorithms discussed below) or portions thereof and/or a signal processing algorithm and/or physiological algorithm or other control functions and/or parameters.

In an additional (envisioned) embodiment of the invention, the ear adapter 60 includes at least one balloon with a pre-set gas pressure. According to the invention, the balloon can be disposed within the adapter housing 61 of the cover 64 and/or base 62.

In another envisioned embodiment, the ear adapter 60 includes one or more inflatable gas-tight bags. According to the invention, the gas-tight bags can be filled with a gas, such as air or argon, or a suitable, non-toxic, patient safe, stable liquid, such as water, isopropanol, silicon fluid, perfluorinated hydrocarbon, etc.

As will be readily appreciated by one having ordinary skill in the art, each of the noted envisioned embodiments would allow an investigator to regulate the pressure exerted on the ear 6 with adapter 60, and, hence, minimize venous effects. Further, in direct analogy to the standard of care associated with cuff devices for measuring blood pressure, as the bag pressure, and with that the pressure on the sensing site, such as the ear lobe, is increased to that of the diastolic and thereafter to the systolic level, the resulting change in the optical signal amplitude provides the relevant indication of peripheral diastolic, systolic and calculated mean arterial pressure.

In the envisioned inflatable gas-tight bag embodiment, the ear adapter 60 preferably includes at least one pressure sensor that is designed and positioned to monitor the applied pressure and an ear pressure algorithm that is adapted to regulate the pressure exerted on the ear 6. The ear pressure algorithm would similarly be included in the processor means 50 and/or monitor.

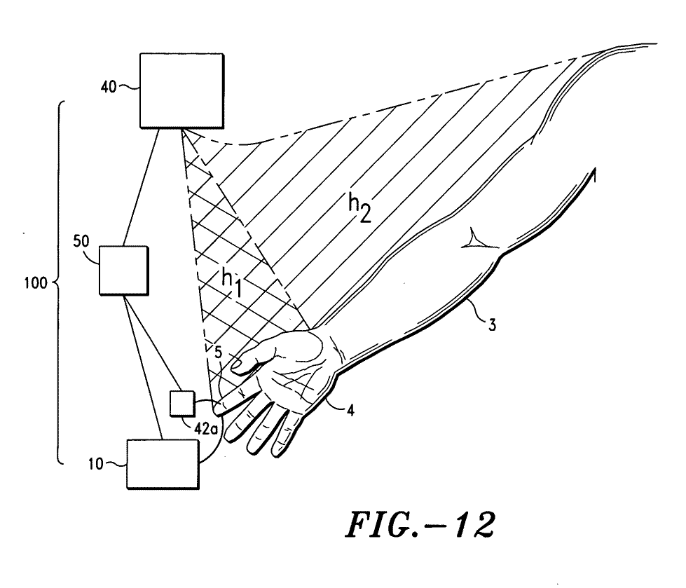

Referring now to FIG. 11, in another envisioned embodiment of the invention, the ear clip 80 includes a bladder 85 that can be similarly filled with a gas or suitable liquid to regulate the pressure applied exerted on the ear lobe 7. In these embodiments, the ear clip 80 preferably similarly includes at least one pressure sensor and the processor would include a further pressure algorithm, i.e. ear lobe pressure algorithm, which is adapted to control the pressure exerted on the ear lobe 7.

Referring now to FIG. 12, there is shown a schematic illustration of the application of heat to a hand 4 (shown as heat zone “h₁”) or alternatively, the entire arm 3 (shown as heat zone “h₂”) by heating means 40. According to the invention, the heat can be applied to the hand 4 and/or arm 3 prior to or in conjunction with obtaining an oximeter (and/or other physiological) reading on a site therein.

As discussed in detail below, the heat can also be applied to a digit or finger 5 prior to or in conjunction with obtaining an oximeter (and/or other physiological) reading on a site therein.

Referring back to FIG. 12, temperature sensor 42a is preferably disposed proximate the heated finger 5. However, the temperature sensor 42a can also be readily disposed proximate any desired location within heat zone “h₁” and, hence, hand 4 or heat zone “h₂” and, hence, arm 3. According to the invention, two or more temperature sensors 42a and/or 42b (discussed above) can also be employed. For example, during heating of the entire arm, one temperature sensor 42a can be disposed proximate a location on the heated arm 3 and one temperature sensor 42a can be disposed proximate the heated hand 4 or finger 5. During heating of the hand 4, one temperature one temperature sensor 42a can be disposed proximate the heated hand 4 or finger 5 and one sensor 42b can be disposed proximate a location on the unheated heated arm 3, i.e. a location that is independent of the heated hand, to monitor the body's response to the heat stimuli.

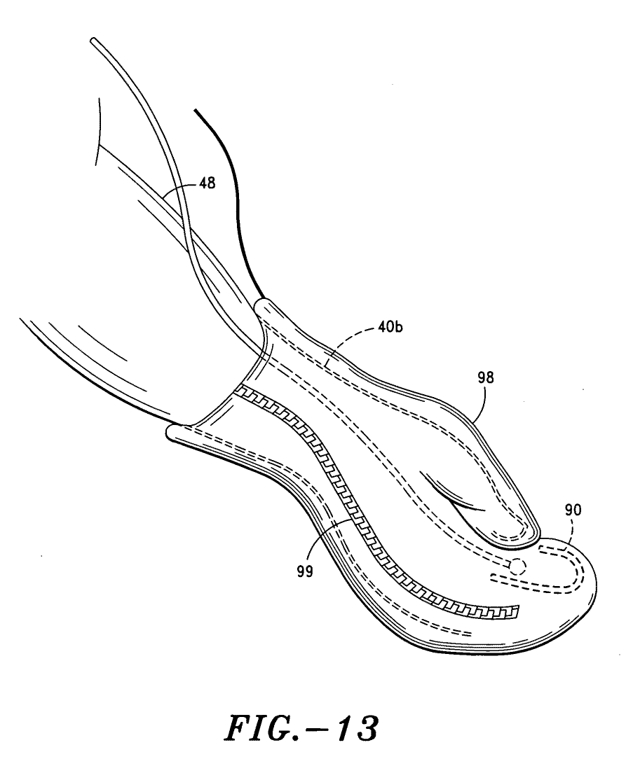

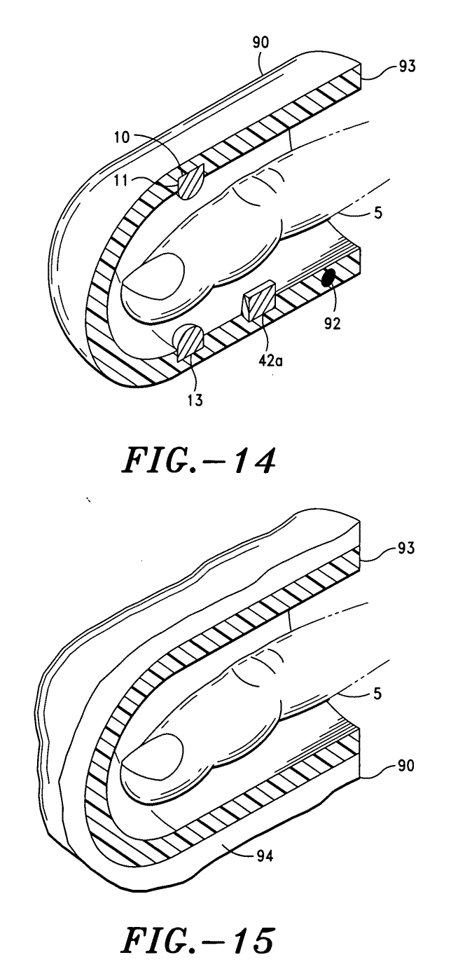

Referring now to FIGS. 13 and 14, there is shown one embodiment of a finger adapter 90 of the invention, which, in the illustrated embodiment, is positioned in a hand warmer (or glove) 98 (i.e. peripheral circulation system).

According to the invention, the hand warmer 98 is designed to encase the hand 4 and, preferably, a portion of the arm 3 (more preferably, the wrist). The hand warmer 98 is preferably constructed of a light weight, insulating material, such as the medical grade, closed cell foam, i.e. DSP0018, distributed by Diversified Silicone Products, Inc.

In the illustrated embodiment, the hand warmer 98 includes closure means 99 to facilitate proper engagement to and positioning on the hand 4, and clinical access. According to the invention, the closure means 99 can comprise any conventional means, such as snap and Velcro systems, and a zipper.

Preferably, the hand warmer 98 includes heating means 40b. According to the invention, the heating means 40b can similarly comprise any of the aforementioned heating means, such as radiative, conductive and convective heating means. In one embodiment of the invention, the hand warmer heating means 40B comprises a flex circuit heater, such as the Minco PDF Heater.

Referring now to FIG. 14, the finger adapter 90 is designed and configured to encase a designated finger 5. As illustrated in FIG. 14, the finger adapter 90 includes a housing 93 that is preferably constructed of light weight material having sufficient rigidity. As will be appreciated by one having ordinary skill in the art, finger adapter housing 93 can comprise various materials. In one embodiment of the invention, the housing 93 is constructed of closed cell polyethylene foam, such as TM3201 medical foam, distributed by MACtac.

As further illustrated in FIG. 14, the finger adapter 90 similarly includes at least one optical interrogating means, such as oximetry sensor 10, having a light source 11 and detector 13. The finger adapter 90 further includes at least one tissue temperature sensing means, such as temperature sensor 42A, for monitoring the temperature of the finger 5, and a finger adapter sensor 92 for monitoring the temperature of the finger adapter housing 93.

In a preferred embodiment of the invention, the hand warmer heating means 42b, oximetry sensor 10 and temperature sensors 42a, 92 are in communication with processor means 50, which includes at least one finger temperature algorithm that is similar to the ear and ear lobe temperature algorithms discussed above and/or at least one of the aforementioned signal processing algorithms and/or physiological algorithms.

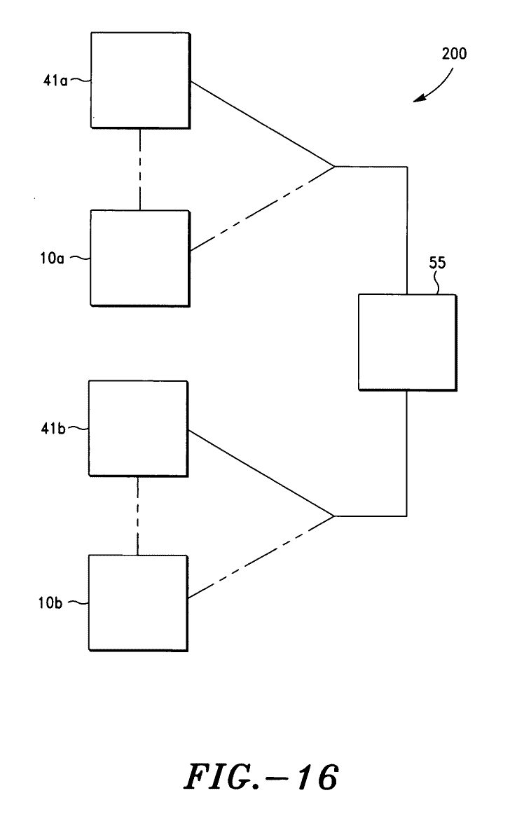

Referring now to FIG. 15, in another embodiment of the invention, the finger adapter 90 similarly includes a balloon or inflatable gas-tight bag 94 that can be filled with a gas or suitable liquid. According to the invention, the balloon and bag would function in a manner that is similar to the envisioned ear adapter balloon and bag(s) discussed above.

In the noted embodiment, the finger adapter 90 preferably includes at least one finger adapter pressure sensor that is adapted to monitor the pressure exerted on the finger 5. Preferably, the processor means 50 includes a finger pressure algorithm similar to the ear and ear lobe pressure algorithms, discussed above.

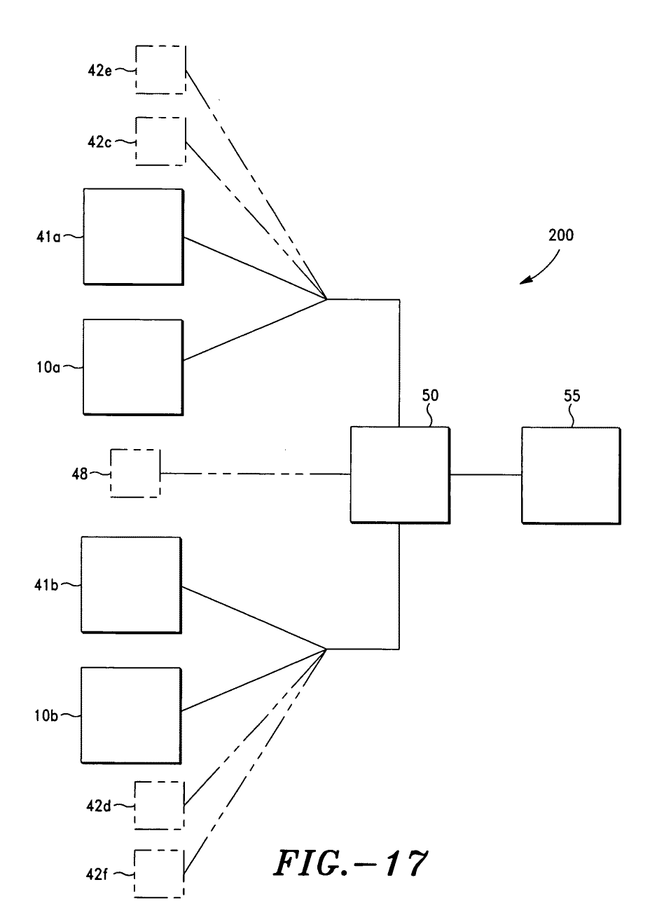

Referring now to FIG. 16, there is shown a schematic illustration of another embodiment of an integrated physiological sensor apparatus and system of the invention (denoted generally “200”). As illustrated in FIG. 16, the system 200 includes a plurality of sensors 10a, 10b. According to the invention, the sensors 10a, 10b can be similar or comprise different sensors, e.g., different physiological measurements, physical dimensions, attachment means, tuning, etc. Thus, in one embodiment of the invention, at least one sensor, i.e. 10a or 10b, is similar to sensor 10.

According to the invention, each physiological sensor 10a, 10b is adapted to be disposed proximate a desired position of the body, e.g., earlobe and finger, and obtain a physiological measurement, such as an oximetry reading, therefrom. In one embodiment of the invention (discussed below), each sensor 10a, 10b comprises an oximetry sensor, wherein at least one sensor, e.g., 10a, is disposed proximate a central circulation site, e.g., neck, ear, nose, etc., and at least one sensor, e.g., 10b, is disposed proximate a peripheral circulation site, e.g., arm, hand, finger, etc.

The system 200 also includes a plurality of associated heating means 41a, 41b, which are similarly adapted to transmit heat energy to a large tissue region, i.e. a tissue region that extends beyond the respective sensor position or target measurement site and/or the tissue region that is proximate to or in direct communication with the respective sensor, prior to or in conjunction with obtaining an oximeter readings, and, optionally, display 55. The heating means 41a, 41b are similarly adapted to be positioned proximate desired locations on the body and transmit heat or heat energy thereto.

As will be readily appreciated by one having ordinary skill in the art, each (or both) heating means 41a, 41b of the invention can also be adapted to heat a smaller tissue region, e.g., a tissue region proximate a respective sensor, if desired.

According to the invention, heating means 41a can be similar to heating means 41b, e.g., heat lamp, or, alternatively, heating means 41a, 41b can comprise different heat sources, e.g., heat lamp, heat blanket and passive heating means. As is also illustrated in FIG. 15, each heating means 41a, 41b can similarly be in communication with a respective sensor 10a, 10b and/or the display 55, whereby the heat transmitted by the heating means 41aA and/or 41b can be displayed and, hence, monitored.

Although system 200 is shown with two physiological sensors, i.e. sensors 10a, 10b, and associated heating means 41a, 41b, it is to be understood that system 200 can include more than two sensors with associated heating means, e.g. three, four, etc. The illustration of system 200 in FIG. 15 (and FIG. 16, discussed below) should thus not be deemed limiting in any manner.

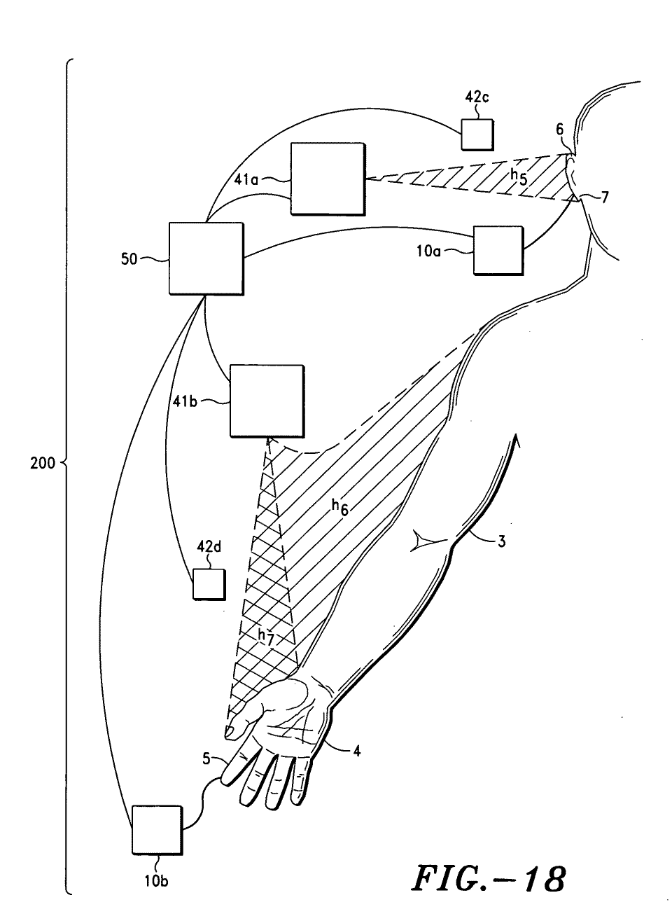

Referring now to FIG. 17, in some embodiments, the system 200 similarly includes processor means (or processor) 50 that is in communication with heating means 41a, 41b, sensors 10a, 10b and display 55, and is programmed and adapted to regulate heating means 41a, 41b and/or sensors 10a, 10b and/or the output displayed on display 55.

In yet additional embodiments, the system 200 includes at least two temperature sensors 42c, 42d that are similarly adapted to be disposed proximate the heated tissue regions and monitor the temperature thereof. Preferably, the temperature sensors 42c, 42d are preferably in communication with the processor 50 and, hence, display 55, whereby the temperature of at least one heated tissue region can be displayed.

As illustrated in FIG. 17, the system includes at least one, preferably, a second pair of temperature sensors 42e, 42f that are also preferably in communication with processor means 50; each sensor 42e, 42f being adapted to be disposed at a body sit that is close to, but independent from the heated tissue region to monitor the temperature thereof and, hence, the body's response to the heat stimuli.

In a preferred embodiment of the invention, the system 200 includes at least one of the aforementioned temperature algorithms that is tailored to the respective body site, e.g., ear, ear lobe or finger, and adapted to regulate the temperature provided by the heating means, i.e. 41a and/or 41b.

In a further embodiment of the invention, the system 200 similarly includes an ECG sensor (shown in phantom and designated “48”) that is adapted to monitor electrical signals and/or impulses associated with heart function.

In some embodiments of the invention, the system 200 further includes at least one additional physiological sensor that is adapted to monitor a further physiological characteristic, such as blood pressure.

Referring now to FIG. 18, there is shown one application of system 200, where one sensor 10a is positioned proximate to and in communication with an ear lobe 7 and one sensor 10b is positioned proximate to and in communication with a finger 5. As illustrated in FIG. 18, heating means 41a is also preferably positioned proximate the ear 6, where heating of the entire ear 6 (shown as heat zone “h₅”) or the ear 6 and adjoining tissue region is possible, if desired. Heating means 41b is preferably positioned proximate the arm 3 and hand 4, where heating of the arm 3 (shown as heat zone “h₆”) and/or hand 4 (shown as heat zone “h₇”) is possible, if desired.

According to the invention, one or both regions, e.g., ear 6 and arm 3, can be heated while obtaining oximetry readings with sensors 10a, 10b. Thus, in one embodiment of the invention, the entire ear 6 (or the ear 6 and adjoining tissue region) is heated with heating means 41a while oximeter readings are acquired at the heated earlobe 7 and the unheated finger 5 with sensors 10a and 10b, respectively. In another embodiment, the entire arm 3 is heated with heating means 41b while oximeter readings are acquired at the unheated ear lobe 7 and heated finger 5 with sensors 10a and 10b, respectively. In yet another embodiment, the hand 4 is heated with heating means 41b while oximeter readings are acquired at the unheated ear lobe 7 and heated finger 5 with sensors 10a and 10b, respectively. In yet another embodiment, the entire ear 6 (or the ear 6 and adjoining tissue region) is heated with heating means 41a and the hand 4 is heated with heating means 41b while oximeter readings are acquired at the heated ear lobe 7 and heated finger 5 with sensors 10a and 10b, respectively.

According to the invention, physiological measurements, e.g., oximetry readings, can also be obtained with sensors 10a, 10b (and any other employed physiological sensor) without the application of heat to an extended tissue region or during (or after a predetermined time after) the application of heat to a smaller tissue region proximate one or both sensors 10a, 10b.

System 200 thus provides an effective means of acquiring multiple oximetry readings with enhanced accuracy from sensors disposed at multiple locations on the body.

According to the invention, an exemplar integrated physiological-sensor would thus comprise a system having both the central circulation system i.e., ear adapter 60, and peripheral circulation system, i.e., hand warmer 98/finger adapter 90, discussed above.

### EXAMPLES

The following examples are provided to enable those skilled in the art to more clearly understand and practice the present invention. They should not be considered as limiting the scope of the invention, but merely as being illustrated as representative thereof.

### Example 1

A series of blood oximetry readings were obtained from thirty-three (33) subjects that ranged in age from 28 to 92 years of age. Baseline temperature and plethysmographic readings were initially recorded. The baseline temperature for each subject was obtained on an area of the ear proximate the sensor using a remote IR skin temperature monitoring device. Baseline plethysmographic recordings were obtained with a non-heatable Nellcor Ear Sensor®, model ES-3212-9.

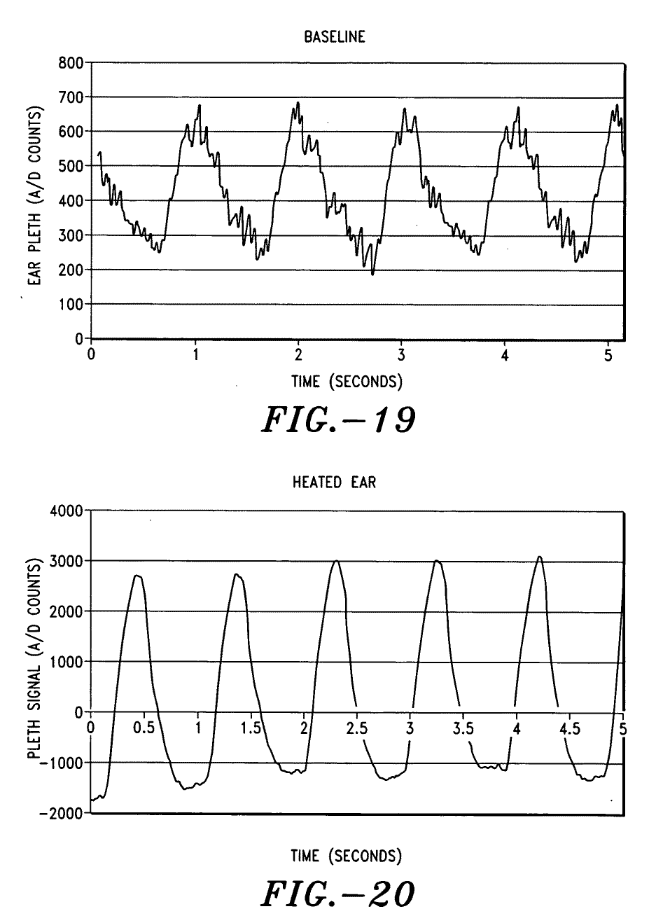

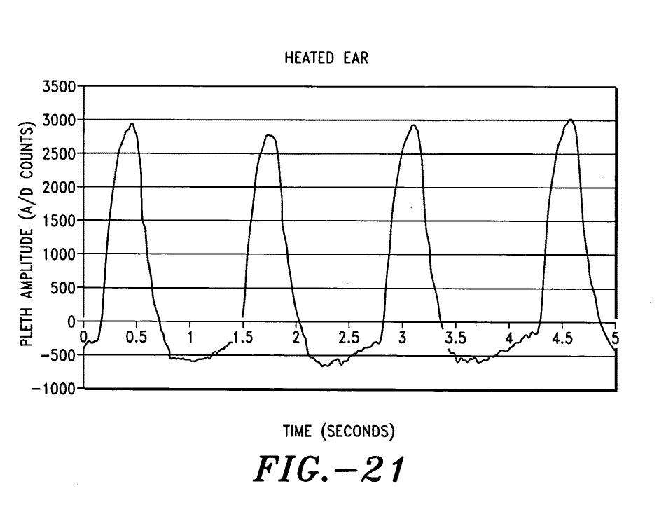

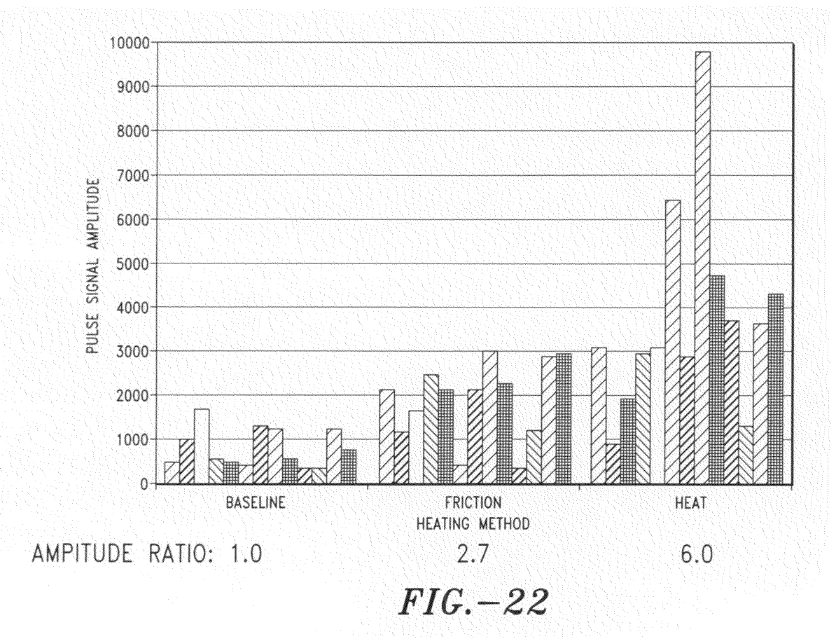

Referring now to FIGS. 19-21, there are shown the IR portions of oximetry plethysmograms obtained on an area of the ear at a baseline temperature in the range of approximately 29-32° C. (FIG. 19) and at an elevated temperature in the range of approximately 35-37° C. for two subjects (FIGS. 20 and 21). It can be seen that the signal-to-noise ratio of the sensor is substantially improved in FIGS. 19 and 20 (i.e. elevated temperature), as evidenced by the absence of the spikes associated with the pulse waves at the baseline temperatures (i.e. FIG. 19).

It should further be noted that the amplitude of the pulse waves shown in FIG. 20 were increased from approximately 400 units (A/D counts) to approximately 3900 units, which reflects a substantial increase of approximately one order of magnitude.

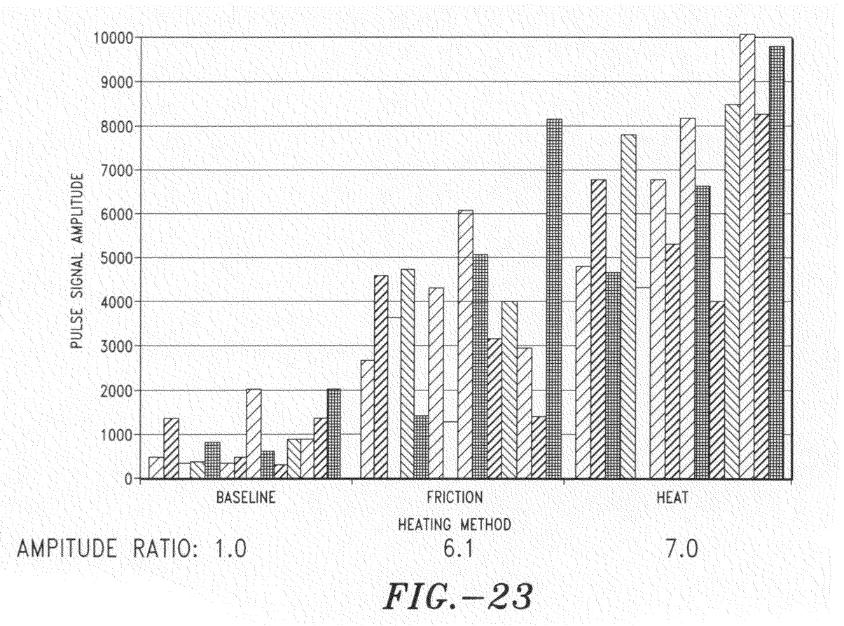

Referring now to FIG. 22; there is shown the effect of different heating methods or conditions for subjects ranging in age from 71-94 years of age on pulse amplitude (or signal). The heating methods or conditions comprised heating the ear to a temperature in the range of approximately 33-35° C. via “friction”, i.e. rubbing the earlobe for approximately 30 seconds, and active (or contact) heating, referred to as “heat” to a temperature of approximately 35-37° C. via a heater blanket.

As illustrated in FIG. 22, heating to a temperature of approximately 33-35° C. via “friction” produced an average 2.7-fold improvement in the amplitude ratio. Contact heating produced an average 6-fold improvement in the amplitude ratio.

Referring now to FIG. 23, there is shown the effect of the same heating methods for subjects ranging in age from 25-26 years of age on the pulse amplitude. As illustrated in FIG. 23, “friction” heating produced an average 6.1-fold improvement in the amplitude ratio. Contact heating produced an average 10.7-fold improvement in the amplitude ratio.

The data reflected in FIGS. 19-23 thus demonstrates that significant improvements in the signal-to-noise ratio of a sensor and, hence, the accuracy of physiological characteristics determined therefrom, can be obtained by virtue of the methods and systems of the invention.

As will readily be appreciated by one having ordinary skill in the art, the physiological sensor methods and systems of the invention provide numerous advantages. Among the advantages are the following:
The provision of physiological sensor apparatus, systems and methods that enhance the accuracy of physiological measurements and determinations made therefrom.
The provision of physiological sensor apparatus, systems and methods that enhance the accuracy of blood parameter determinations of oximetry sensors, such as oxygen saturation.
The provision of physiological sensor apparatus, systems and methods that can readily be incorporated in or employed in conjunction with conventional oximetry sensors to enhance the accuracy of blood parameter readings and/or determinations made therefrom.
The provision of physiological sensor apparatus, systems and methods that facilitate the acquisition of signals reflecting physiological characteristic at a body or tissue site that is supplied by the central circulation, such as a site on the head, and/or allows for monitoring of patients that are peripherally vasoconstricted to the extent that conventional sites, such as a finger or toe, are neither palpable, nor yield usable plethysmographic signals.
The provision of physiological sensor apparatus, systems and methods that facilitate the acquisition of signals reflecting physiological characteristic at a site that is proximate the aorta where the wave shape is much less influenced by transit through vasculature of complex shape, branching and length at a patient-dependent degree of hardening of the arterial wall. Thus, the pressure and flow wave shape is more similar to the original shape as it leaves the aorta, which enables accurate measurements and diagnostic information of hemodynamic parameters, such as blood pressure, cardiac output, structure condition and functioning of the arterial vasculature.
The provision of physiological sensor apparatus, systems and methods that provide heating at a constant or variable rate to a set temperature and monitoring of amplitudes or time changes of the arterial pressure induced signals, whereby the pressure or flow waveforms yields information on the degree of physiological control of that patient, as well as indirectly on therapeutic or otherwise interventional effectiveness.
The provision of physiological sensor apparatus, systems and methods that include thermal control of the measurement site, whereby the effects of temperature variability or fluctuation, and/or the body's response to the heat stimuli on the measured parameter(s), e.g. oxygen saturation, is minimized.
The provision of physiological sensor apparatus, systems and methods that include heating means, thermal control of the heating means and measurement site, and a plurality of physiological sensors, e.g., oxygen saturation, blood pressure, ECG, etc.

Without departing from the spirit and scope of this invention, one having ordinary skill in the art can make various changes and modifications to the invention to adapt it to various usages and conditions. As such, these changes and modifications are properly, equitably, and intended to be, within the full range of equivalence of the following claims.

What is claimed is:

1. An integrated physiological sensor system, comprising:
a plurality of physiological sensors, said plurality of physiological sensors including at least a first physiological sensor adapted to measure pulse amplitude at a target measurement site on a subject's body and a second physiological sensor adapted to monitor electrical impulses associated with said subject's heart function; and
means for heating a tissue region on said subject's body, whereby blood perfusion of said tissue region is enhanced, said tissue region including said target measurement site and extending beyond said target measurement site.

2. The system of claim 1, wherein said system includes a third sensor adapted to monitor blood pressure.

3. An integrated physiological sensor system, comprising:
a plurality of physiological sensors, said plurality of sensors including at least a first physiological sensor adapted to measure pulse amplitude at a first target measurement site on a subject's body and a second physiological sensor adapted to monitor electrical impulses associated with said subject's heart function;
means for heating a tissue region on said subject's body, whereby blood perfusion of said tissue region is enhanced, said tissue region including said first target measurement site and extending beyond said first target measurement site;
a first heat sensor adapted to monitor skin surface temperature of said tissue region; and
a processor in communication with said heating means and said first heat sensor, said processor including at least one algorithm for regulating said heating means based on a physiological response of said subject's body to heating of said tissue region.

4. The system of claim 1, wherein said first physiological sensor comprises a pulse oximetry sensor, said pulse oximetry sensor having a signal-to noise ratio.

5. The system of claim 4, wherein said processor includes stored demographic pulse amplitude and skin surface temperature data, and wherein said algorithm is adapted to compare first pulse amplitude measured by said first physiological sensor at said first target measurement site and first skin surface temperature of said tissue region measured by said first heat sensor to said stored demographic amplitude and skin surface temperature data, and adjust the heat provided by said heating means based on said comparison, whereby said signal-to-noise ratio of said first physiological sensor is optimized.

6. The system of claim 5, wherein said demographic pulse amplitude and skin surface temperature data includes measured pulse amplitudes and skin surface temperatures of a second tissue region on a plurality of second subjects during heating of a first tissue region on said second subjects' body, said second subjects' second tissue region being close to, but independent of said second subjects' first tissue region.

7. The system of claim 4, wherein said system includes a third physiological sensor adapted to measure pulse amplitude at a second target measurement site that is close to said first target measurement site, but independent thereof, and a second heat sensor adapted to monitor skin surface temperature of said second target measurement site, said third physiological sensor and said second heat sensor being in communication with said processor.

8. The system of claim 7, wherein said algorithm is adapted to compare first pulse amplitude measured by said first physiological sensor at said first target measurement site and first skin surface temperature of said tissue region measured by said first heat sensor to second pulse amplitude measured at said second target measurement site by said third physiological sensor and second skin temperature of said second target measurement site measured by said second heat sensor, and adjust the heat provided by said heating means base on said comparison, whereby said signal-to-noise ratio of said first physiological sensor is optimized.

9. The system of claim 1, wherein at least said first physiological sensor includes at least one lead operatively connected to said first physiological sensor and said processor to facilitate communication by and between said first physiological sensor and said processor, said first physiological sensor lead including quick-disconnect means.

10. The system of claim 9, wherein said processor further includes a disconnect algorithm that is adapted to monitor said quick-disconnect means and limit operation of said system after a predetermined number of subsequent re-connections of said quick-disconnect means after a predetermined period of time.

11. The system of claim 1, wherein said system includes at least one ear adapter adapted to engage an ear of said subject, said ear adapter including said first physiological sensor.

12. The system of claim 11, wherein said ear adapter further includes means for applying pressure to the ear lobe of said engaged ear and at least one pressure sensor adapted to monitor said applied pressure on said ear lobe.

13. The system of claim 12, wherein said processor further includes an ear pressure algorithm adapted to regulate said applied pressure on said ear lobe.

14. The system of claim 1, wherein said system includes a fourth sensor adapted to monitor blood pressure.

15. A method of determining a physiological characteristic, comprising the steps of:
providing at least a first physiological sensor that is adapted to measure pulse amplitude at a first tissue region on a subject's body, said first physiological sensor having a signal-to-noise ratio;
disposing said first physiological sensor proximate said first tissue region;
heating said first tissue region to an interrogation temperature;
measuring a first pulse amplitude at said first tissue region with said first physiological sensor during heating of said first tissue region;
measuring a first temperature of said first tissue region during heating of said first tissue region;
providing demographic pulse amplitude and skin surface temperature data;
providing a temperature algorithm that is adapted to adjust the interrogation temperature as a function of said first pulse amplitude and said first tissue region temperature and said demographic pulse,amplitude and skin surface temperature data, whereby said signal-to-noise ratio of said first physiological sensor is optimized.

16. The method of claim 15, wherein said demographic pulse amplitude and skin surface temperature data includes measured pulse amplitudes and skin surface temperatures of a second tissue region on a plurality of second subjects during heating of a first tissue region on said second subjects' body, said second subjects' second tissue region being close to, but independent of said second subjects' first tissue region.

17. The method of claim 15, wherein said heating of said first tissue region is sufficient to induce an optimal homeostatic reflex, whereby said first issue region blood perfusion is enhanced, without burning said subject.

18. The method of claim 15, including the step of monitoring electrical impulses associated with said subject's heart function with a second physiological sensor.

19. The method of claim 15, including the step of monitoring blood pressure with a third physiological sensor.

20. A method of determining a physiological characteristic, comprising the steps of:
providing a first physiological sensor that is adapted to measure pulse amplitude at a first tissue region on a subject's body, said first physiological sensor having a signal-to-noise ratio;
disposing said first physiological sensor proximate said first tissue region;
providing a second physiological sensor that is adapted to measure pulse amplitude at a second tissue region on said subject's body, said second tissue region being close to, but independent of said first tissue region;
disposing said second physiological sensor proximate said second tissue region;
heating said first tissue region to an interrogation temperature;
measuring a first temperature of said first tissue region during heating of said first tissue region;
measuring a first pulse amplitude at said first tissue region with said first physiological sensor during heating of said first tissue region;
measuring a second temperature of a second tissue region;
measuring a second pulse amplitude at said second tissue region with said second physiological sensor;
providing an algorithm that is adapted to adjust said interrogation temperature as a function of said first and second tissue region temperatures and said first and second pulse amplitudes, whereby said signal-to-noise ratio of said first physiological sensor is optimized.

21. The method of claim 20, wherein said heating of said first tissue region is sufficient to induce an optimal homeostatic reflex, whereby said first issue region blood perfusion is enhanced, without burning said subject.

22. The method of claim 20, including the step of monitoring electrical impulses associated with said subject's heart function with a second physiological sensor.

23. The method of claim 20, including the step of monitoring blood pressure with a third physiological sensor.
